(19) United States
(12) Patent Application Publication
(10) Pub. No.: US 2011/0060362 A1
(43) Pub. Date: Mar. 10, 2011

(54) DEVICES AND METHODS FOR TREATING RECTOVAGINAL AND OTHER FISTULAE

(76) Inventors: Umesh H. Patel, West Lafayette, IN (US); F. Joseph Obermiller, West Lafayette, IN (US)

(21) Appl. No.: 12/948,177

(22) Filed: Nov. 17, 2010

Related U.S. Application Data
(63) Continuation of application No. PCT/US2009/045467, filed on May 28, 2009.
(60) Provisional application No. 61/057,072, filed on May 29, 2008.

(51) Int. Cl. A61B 17/03

(52) U.S. Cl. 606/215

(57) ABSTRACT

Provided by certain aspects of the invention are methods for treating fistulae and various fistula graft assemblages useful in this regard. Illustratively, some inventive methods are useful in treating fistulae having at least a first fistula opening, a second fistula opening and a fistula tract extending therebetween. In one step, a first capping member is positioned over the first fistula opening such that a first pulling member, which extends from the first capping member, passes through the fistula tract. In another step, a second capping member is positioned over the second fistula opening such that a second pulling member, which extends from the second capping member, passes through the fistula tract. A first pulling force is applied to the first pulling member, and a second pulling force is applied to the second pulling member, for maintaining the first capping member over the first fistula opening and for maintaining the second capping member over the second fistula opening, respectively.

### REFERENCE TO RELATED APPLICATIONS

This application is a continuation of International Application No. PCT/US2009/045467 filed May 28, 2009, which claims the benefit of U.S. Provisional Application No. 61/057,072 filed May 29, 2008, each of which is hereby incorporated by reference.

### BACKGROUND

The present invention relates generally to medical technology and in particular aspects to devices and methods for treating fistulae.

As further background, a variety of fistulae can occur in humans. These fistulae can occur for a variety of reasons, such as but not limited to, as a congenital defect, as a result of inflammatory bowel disease, such as Crohn's disease, irradiation, trauma, such as childbirth, or as a side effect from a surgical procedure. Further, several different types of fistulae can occur, for example, urethro-vaginal fistulae, vesico-vaginal fistulae, tracheo-esophageal fistulae, gastro-cutaneous fistulae, and any number of anorectal fistulae, such as recto-vaginal fistula, recto-vesical fistulae, recto-urethral fistulae, or recto-prostatic fistulae.

Anorectal fistulae can result from infection in the anal glands, which are located around the circumference of the distal anal canal that forms the anatomic landmark known as the dentate line. Approximately 20-40 such glands are found in humans. Infection in an anal gland can result in an abscess. This abscess then can track through soft tissues (e.g., through or around the sphincter muscles) into the perianal skin, where it drains either spontaneously or surgically. The resulting void through soft tissue is known as a fistula. The internal or inner opening of the fistula, usually located at or near the dentate line, is known as the primary opening. Any external or outer openings, which are usually located in the perianal skin, are known as secondary openings.

The path which these fistulae take, and their complexity, can vary. A fistula may take a take a “straight line” path from the primary to the secondary opening, known as a simple fistula. Alternatively, the fistula may consist of multiple tracts ramifying from the primary opening and have multiple secondary openings. This is known as a complex fistula.

The anatomic path which such fistulae take is classified according to their relationship to the anal sphincter muscles. The anal sphincter consists of two concentric bands of muscle, the inner or internal sphincter and the outer or external anal sphincter. Fistulae which pass between the two concentric anal sphincters are known as inter-sphincteric fistulae. Those which pass through both internal and external sphincters are known as trans-sphincteric fistulae, and those which pass above both sphincters are called supra-sphincteric fistula. Fistulae resulting from Crohn's disease usually “ignore” these anatomic planes, and are known a “extra-anatomic” fistulae.

Many complex fistulae consist of multiple tracts, some blind-ending and others leading to multiple secondary openings. One of the most common complex fistulae is known as a horseshoe fistula. In this instance, the infection starts in the anal gland (primary opening) at or near the 12o'clock location (with the patient in the prone position). From this primary opening, fistulae pass bilaterally around the anal canal, in a circumferential manner. Multiple secondary openings from a horseshoe fistula may occur anywhere around the periphery of the anal canal, resulting in a fistula tract with a characteristic horseshoe configuration.

One technique for treating a perianal fistulae is to make an incision adjacent the anus until the incision contacts the fistula and then excise the fistula from the anal tissue. This surgical procedure tends to sever the fibers of the anal sphincter, and may cause incontinence.

Other surgical treatment of fistulae involve passing a fistula probe through the tract of the fistula in a blind manner, using primarily only tactile sensation and experience to guide to probe. Having passed the probe through the fistula tract, the overlying tissue is surgically divided. This is known as a fistulotomy. Since a variable amount of sphincter muscle is divided during the procedure, fistulotomy also may result in impaired sphincter control, and even frank incontinence.

Still other methods involve injecting sclerosant or sealant (e.g., collagen or fibrin glue) into the tract of the fistula to block the fistula. Closure of a fistula using a sealant is typically performed as a two-stage procedure, including a first-stage seton placement and injection of the fibrin glue several weeks later. This allows residual infection to resolve and to allow the fistula tract to “mature” prior to injecting a sealant. If sealant or sclerosant were injected as a one-stage procedure, into an “unprepared” or infected fistula, this may cause a flare-up of the infection and even further abscess formation.

A gastrointestinal fistula is an abnormal passage that leaks contents of the stomach or the intestine (small or large bowel) to other organs, usually other parts of the intestine or the skin. For example, gastrojejunocolic fistulae include both enterocutaneous fistulae (those occurring between the skin surface and the intestine, namely the duodenum, the jejunum, and the ileum) and gastric fistulae (those occurring between the stomach and skin surface). Another type of fistula occurring in the gastrointestinal tract is an enteroenteral fistula, which refers to a fistula occurring between two parts of the intestine. Gastrointestinal fistulae can result in malnutrition and dehydration depending on their location in the gastrointestinal tract. They can also be a source of skin problems and infection. The majority of these types of fistulae are the result of surgery (e.g., bowel surgery), although sometimes they can develop spontaneously or from trauma, especially penetrating traumas such as stab wounds or gunshot wounds. Inflammatory processes, such as infection or inflammatory bowel disease (Crohn's disease), may also cause gastrointestinal fistulae. In fact, Crohn's disease is the most common primary bowel disease leading to enterocutaneous fistulae, and surgical treatment may be difficult because additional enterocutaneous fistulae develop in many of these patients postoperatively.

Treatment options for gastrointestinal fistulae vary. Depending on the clinical situation, patients may require IV nutrition and a period of time without food to allow the fistula time to close on its own. Indeed, nonsurgical therapy may allow spontaneous closure of the fistula, although this can be expected less than 30% of the time according to one estimate. A variable amount of time to allow spontaneous closure of fistulae has been recommended, ranging from 30 days to 6 to 8 weeks. During this preoperative preparation, external control of the fistula drainage prevents skin disruption and provides guidelines for fluid and electrolyte replacement. In some cases, surgery is necessary to remove the segment of intestine involved in a non-healing fistula.

There remain needs for improved and/or alternative devices and methods for treating fistulae including rectovaginal fistulae. The present invention is addressed to those needs.

### SUMMARY

The present invention provides, in certain aspects, unique products for treating fistulae, for example, those having at least a first fistula opening, a second fistula opening and a fistula tract extending therebetween. One illustrative product is a fistula graft assembly that includes a first capping member, a second capping member and an elongate plug body. Also included in the assembly are a first pulling member and a second pulling member, which extend from the first capping member and the second capping member, respectively. Each of the various assembly components can exhibit a variety of shapes and sizes, and each may be formed with one or more of a variety of materials. In this regard, the plug member may or may not be formed with the same material as one or both capping members. In some forms, all or part of an assembly is formed with a collagen-containing material, for example, a remodelable extracellular matrix (ECM) material. Illustratively, the plug body may be formed with an ECM material, while one or both capping members may be formed with a synthetic polymeric material or other non-ECM material. The first capping member and second capping member are configured to be positioned over the first fistula opening and the second fistula opening, respectively, while the elongate plug body is configured to reside in the fistula tract. The first pulling member and the second pulling member are configured to extend along the plug body in the fistula tract, and can be contemporaneously pulled and in generally opposite directions for maintaining the first capping member over the first fistula opening and the second capping member over the second fistula opening. With the pulling members tensioned, the assembly can be generally fixed in this tensioned state so that the assembly remains seated in the fistula tract, for example, by tying off the pulling members on the exterior sides of the capping members or by otherwise fixing the pulling members so that the assembly remains tensioned. In some preferred embodiments, an inventive assembly will be secured in a tensioned state without having to attach the assembly to patient tissue.

In another embodiment, the invention provides a fistula graft assembly for treating a fistula having at least a first fistula opening, a second fistula opening and a fistula tract extending therebetween. This assembly includes a first capping member and an elongate plug body. The first capping member is positionable over the first fistula opening, and has a first pulling member extending therefrom. The elongate plug body is configured to reside in the fistula tract, and includes a portion configured to extend through an opening in the first capping member. The first pulling member is configured to extend through the fistula tract, and is pullable in a direction generally away from the first fistula opening for maintaining the first capping member over the first fistula opening.

An additional aspect of the present invention provides a method for treating a fistula having at least a first fistula opening, a second fistula opening and a fistula tract extending therebetween. In this method, a first capping member and a second capping member are provided. Also provided are a first pulling member and a second pulling member, which extend from the first capping member and the second capping member, respectively. In one step, the first capping member is positioned over the first fistula opening such that the first pulling member extends through the fistula tract. In another step, the second capping member is positioned over the second fistula opening such that the second pulling member extends through the fistula tract. A first pulling force is applied to the first pulling member for maintaining the first capping member over the first fistula opening. Contemporaneous with the application of the first pulling force, a second pulling force is applied to the second pulling member for maintaining the second capping member over the second fistula opening.

Another aspect of the invention provides a method of treating a fistula having at least a first fistula opening, a second fistula opening and a fistula tract extending therebetween. This method includes providing (i) an elongate plug body; and (ii) a first capping member having a first pulling member extending therefrom. In one step, the first capping member is positioned over the first fistula opening such that the first pulling member extends through the fistula tract. In another step, the plug body is positioned in the fistula tract, wherein the plug body is advanced through the fistula tract from the second fistula opening and toward the first fistula opening.

Other objects, embodiments, forms, features, advantages, aspects, and benefits of the present invention shall become apparent from the detailed description and drawings included herein.

### BRIEF DESCRIPTION OF THE DRAWINGS

FIG. 1 shows components of an inventive assembly being arranged in and around a fistula.

FIG. 2 shows a capping member according to one embodiment of the invention.

FIG. 3 shows additional components of the assembly of FIG. 1 being arranged in and around a fistula.

FIG. 4 shows still additional components of the assembly of FIGS. 1 and 3 being arranged in and around a fistula.

### DETAILED DESCRIPTION

While the present invention may be embodied in many different forms, for the purpose of promoting an understanding of the principles of the present invention, reference will now be made to the embodiments illustrated in the drawings, and specific language will be used to describe the same. It will nevertheless be understood that no limitation of the scope of the invention is thereby intended. Any alterations and further modifications in the described embodiments and any further applications of the principles of the present invention as described herein are contemplated as would normally occur to one skilled in the art to which the invention relates.

As disclosed above, in certain embodiments, the present invention provides unique products and methods for treating fistulae. Some aspects of the invention involve treating fistulae having at least a first fistula opening, a second fistula opening and a fistula tract extending therebetween. In one illustrative method, a first capping member is positioned over the first fistula opening such that a first pulling member, which extends from the first capping member, passes through the fistula tract. Additionally, a second capping member is positioned over the second fistula opening such that a second pulling member, which extends from the second capping member, passes through the fistula tract. A first pulling force is applied to the first pulling member, and a second pulling force is applied to the second pulling member, for maintaining the first capping member over the first fistula opening and for maintaining the second capping member over the second fistula opening, respectively.

Capping members useful in the invention can be shaped and configured in a variety of manners. In some forms, a capping member will be a thin plate-like object for placement over a fistula opening to fully or partially block the opening. A fistula opening can occur in a bodily tissue wall such as the skin or an alimentary canal wall, and a capping member can be configured to contact portions of the wall adjacent to the opening and remain over the opening. A capping member can include a single object, or alternatively, multiple objects (e.g., pieces of material). While a capping member in one illustrative embodiment is disc-shaped, many other suitably shaped capping members are contemplated as within the scope of the present invention. These include various three-dimensional shapes having rectilinear and/or curvilinear features. Suitable three-dimensional rectilinear shapes can have any suitable number of sides, and can include, for example, cubes, cuboids, tetrahedrons, prisms, pyramids, wedges, and variations thereof. Suitable three-dimensional curvilinear bodies can include, for example, spheres, spheroids, ellipsoids, cylinders, cones, and any suitable variations thereof (e.g., a segment of a sphere, or a truncated cone, etc.).

Capping members useful in the invention can be prepared, for example, as described in International Patent Application Ser. No. PCT/US2006/024260, filed Jun. 21, 2006, and entitled “IMPLANTABLE GRAFT TO CLOSE A FISTULA” (Cook Biotech Incorporated); and International Patent Application Ser. No. PCT/US2007/61371, filed Jan. 31, 2007, and entitled “FISTULA GRAFTS AND RELATED METHODS AND SYSTEMS FOR TREATING FISTULAE” (Cook Biotech Incorporated), which are hereby incorporated by reference in their entirety. These include capping members comprising a resilient wire frame or other similar frame or frame-like support component. In forms where a frame has the capacity to expand, these frames can include those that are considered self-expanding and those that require at least some manipulation in order to expand.

Capping members useful in the invention may be formed with one or more of a variety material including some that are naturally derived and some that are non-naturally derived. In some embodiments, all or part of a capping member will be formed with a suitable synthetic polymeric material including but not limited to bioresorbable and/or non-bioresorbable plastics. Bioresorbable, or bioabsorbable polymers that may be used include, but are not limited to, poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyhydroxyalkanaates, polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, and polyphosphazenes. These or other bioresorbable materials may be used, for example, where only a temporary blocking or closure function is desired, and/or in combination with non-bioresorbable materials where only a temporary participation by the bioresorable material is desired.

Non-bioresorbable, or biostable polymers that may be used include, but are not limited to, polytetrafluoroethylene (PTFE) (including expanded PTFE), polyethylene terephthalate (PET), polyurethanes, silicones, and polyesters and other polymers such as, but not limited to, polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins, polycarbonates; polyoxymethylenes; polyimides; polyethers; epoxy resins, polyurethanes; rayon; and rayon-triacetate.

In some embodiments, an inventive graft assembly will additionally include a plug body that is configured to reside in a fistula tract. A plug body of this sort can exhibit a variety of shapes and sizes, and may be formed with one or more of a variety of materials including some that are naturally derived and some that are non-naturally derived. Although not necessary to broader aspects of the invention, in some aspects, a plug body will be attached to the capping member(s) forming part of the assembly. Additionally, in some forms, a plug body will be configured for positioning in a fistula tract such that it extends toward a capping member, and potentially through an opening in that capping member.

A plug body useful in the invention can be constructed in any suitable manner. In some embodiments, a plug body is formed with a reconstituted or otherwise reassembled ECM material. Forming a plug body may involve folding or rolling, or otherwise overlaying one or more portions of a biocompatible material, such as a biocompatible sheet material. In certain aspects, the overlaid biocompatible sheet material is compressed and dried or otherwise bonded into a volumetric shape such that a substantially unitary construct is formed. In some forms, such a plug body is formed by randomly or regularly packing one or more pieces of single or multilayer ECM sheet material within a mold and thereafter processing the packed material. A suitable plug body can be prepared, for example, as described in International Patent Application Ser. No. PCT/US2006/16748, filed Apr. 29, 2006, and entitled “VOLUMETRIC GRAFTS FOR TREATMENT OF FISTULAE AND RELATED METHODS AND SYSTEMS” (Cook Biotech Incorporated), which is hereby incorporated by reference in its entirety.

When utilized, some elongate plug bodies will be of sufficient size and shape to extend through at least a portion of a fistula tract, and will generally (but not necessarily) be of sufficient dimension to fill a fistula, or a segment thereof, e.g., the primary fistula opening, a fistula tract, and/or any secondary fistula openings, either alone or in combination with other devices. In certain embodiments, an elongate plug body will have a length of at least about 0.20 cm, and in many situations at least about 1 cm to about 20 cm (approximately 1 to 8 inches). In illustrative embodiments, a plug body will have a length of from about 2 cm to about 5 cm, or alternatively, from about 2 inches to about 4 inches. Additionally, in certain embodiments, elongate plug bodies will have a diameter, which may or may not be constant along their length, of from about 0.1 mm to about 25 mm, or more typically from about 5 mm to about 10 mm. In certain embodiments, a generally conical plug body is tapered along its length so that one end of the plug body has a diameter of about 5 mm to about 10 mm and the opposite end of the plug body has a diameter of about 0.5 mm to about 3 mm. Such a taper may or may not be continuous along the length of the elongate plug body.

Additionally or alternatively, a plug body may include a compliant sheet-form material. Such materials can include any of the ECM or other suitable biocompatible materials described herein, for example, a multilaminate ECM material sheet. In preferred embodiments, such a sheet will be deformable upon impingement by soft tissue surrounding a fistula (e.g., tissue surrounding the primary fistula opening, the fistula tract, and/or any secondary fistula openings), and will be sized and shaped so as to be deformable to a three-dimensional volumetric body extending through at least a portion of the fistula tract, and potentially filling at least a portion of the fistula tract, the primary opening, and/or any secondary openings of the fistula. In so doing, advantageous implant materials will also be sufficiently flaccid to avoid substantial cutting or tearing of the surrounding soft tissues. A sheet form plug body can be prepared, for example, as described in U.S. patent application Ser. No. 11/414,682, titled “FISTULA GRAFT WITH DEFORMABLE SHEET-FORM MATERIAL” (Cook Biotech Incorporated) filed Apr. 28, 2006, which is hereby incorporated by reference in its entirety.

A plug body, when included in an inventive graft assembly, may or may not be attached to the capping member(s) present in the device. In this regard, a capping member and an elongate plug body may be formed together as a single unit (e.g., from a single piece of biocompatible material), or alternatively, the two members may be formed separately and then combined with one another, for example, using an adhesive, by suturing, using mechanical fastener(s), and/or any other suitable joining means. When formed separately, the two may or may not be comprised of the same biocompatible material(s). As well, it should be noted that, in certain aspects, a capping member and an elongate plug body are formed from separate pieces of material, yet are retained in association with one another without the use of any other device or material (e.g., sutures, an adhesive, etc.). In such aspects, the capping member and the elongate plug body are held together by having at least one member (or any portion thereof) received around, through, over, etc., the other member (or any portion thereof). In certain preferred aspects, at least part of an inventive graft assembly is formed with a remodelable material such as a remodelable ECM material. Illustratively, a capping member and an elongate plug body can be formed from separate pieces of remodelable SIS material, and thereafter coupled to one another. In some embodiments, one or more capping members are formed with a non-naturally derived material, and a plug body is formed with a naturally derived material.

Products and methods of the invention can be used to treat a variety of fistulae and other passages and openings in the body. In some preferred aspects, products and methods are adapted for treating fistulae having at least a primary opening and a fistula tract extending therefrom, for example, a primary opening in the alimentary canal. Some fistulae to be treated will have at least a first fistula opening, a second fistula opening and a fistula tract extending therebetween. In this context, the term “fistula tract” is meant to include, but is not limited to, a void in soft tissues extending from a primary fistula opening, whether blind-ending or leading to one or more secondary fistula openings, for example, to include what are generally described as simple and complex fistulae.

In this regard, inventive products and methods may be useful to treat urethro-vaginal fistulae, vesico-vaginal fistulae, tracheo-esophageal fistulae, gastro-cutaneous fistulae, and any number of anorectal fistulae, such as recto-vaginal fistula, recto-vesical fistulae, recto-urethral fistulae, or recto-prostatic fistulae. Inventive products and methods can be used to treat a fistula regardless of its size and shape, and in some forms, are utilized to treat a fistula having a primary opening, secondary opening, and/or fistula tract with a diameter ranging from about 1 millimeter to about 20 millimeters, more typically from about 5 millimeters to about 10 millimeters.

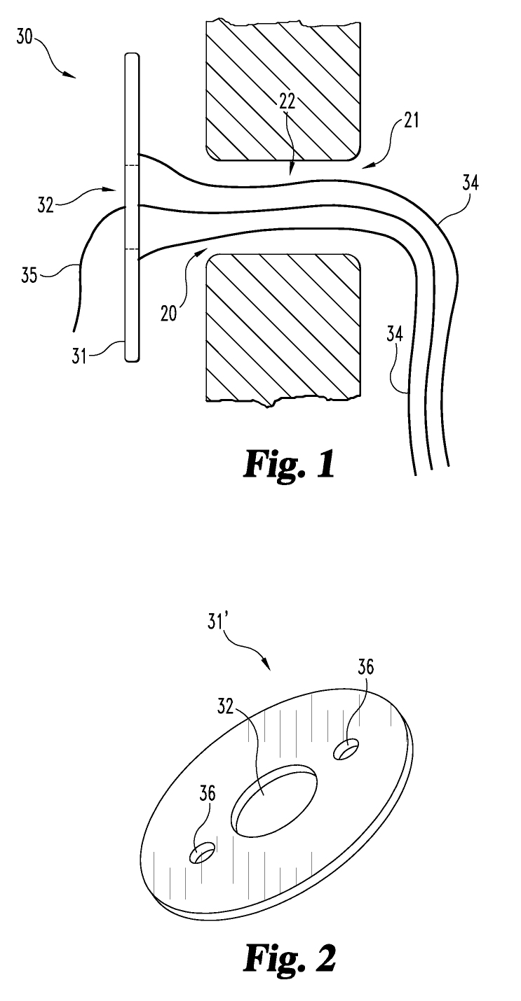

With reference now to FIG. 1, shown is one illustrative manner in which parts of an inventive fistula graft assembly can be arranged in and around a fistula. The fistula includes a first fistula opening 20, a second fistula opening 21 and a fistula tract 22 extending therebetween. An inventive fistula graft assembly 30 includes a first capping member 31 which can be positioned just beyond first fistula opening 20 as shown. A first pulling member 34, which includes a pair of sutures, extends from first capping member 31, and can be passed through fistula tract 22. The sutures can be bonded or coupled to the capping member in a variety of ways. In one embodiment, ends of the sutures are embedded in the capping member. A second pulling member 35 includes a single suture, which can be passed through fistula tract 22 and a central opening 32 in capping member 31. Capping member 31 includes a generally disc-shaped construct formed with a synthetic polymeric material (e.g., Nylon), although such a capping member could be otherwise shaped and configured as described elsewhere herein. The dimensions of the capping member are such that portions of the disc can contact portions of the tissue wall adjacent to first fistula opening 20 when the disc is positioned over the opening as shown.

There are a variety of ways to provide the type of arrangement shown in FIG. 1. One way involves passing a probe or other similar instrument through fistula tract 22 from second fistula opening 21 and toward first fistula opening 20. In some aspects, second pulling member 35 will be releasably attached to this probe for delivery into and through the fistula tract. First pulling member 34 and second pulling member 35 can be coupled to or otherwise associated with the probe, and the probe can be withdrawn back through fistula tract 22, thereby pulling first pulling member 34 and second pulling member 35 therealong. In this regard, first pulling member 34 can be pulled until first capping member 31 is positioned at or near first fistula opening 20. If not already placed, second pulling member 35 can be positioned so that it extends entirely through fistula tract 22 as shown in FIG. 1. The portion of second pulling member 35 extending from second fistula opening 21 can be coupled to another component potentially to be included in the assembly (e.g., a plug body configured to reside in fistula tract 22 and/or a second capping member configured for positioning over second fistula opening 21, etc.). Second pulling member 35 can then be used to pull the additional component(s) into a desirable position in and/or around the fistula to provide treatment.

Manipulation of a pulling member can be performed directly by hand in situations where such access is possible, although in some embodiments, manipulating a pulling member will additionally or alternatively involve the use of one or more instruments. In certain embodiments, manipulating a pulling member can involve the use of a fistula probe or other suitable instrument, for example, an appropriately configured pair of surgical hemostats that include a portion passable through a fistula opening (e.g., a secondary opening), through a fistula tract, and out of another fistula opening (e.g., a primary opening). Thereafter, a pulling member can be releasably grasped by the probe or otherwise coupled to the probe and pulled back through the fistula.

Referring now to FIG. 2, shown is first capping member 31′ which is similar to that depleted in FIG. 1 except that it additionally includes a pair of openings 36 extending through the member. These sorts of openings may be advantageous in certain grafting arrangements, for example, where a suture or other suitable pulling member is secured to the capping member in a manner that involves passing the suture through one or both openings. In addition, this sort of opening can provide an outlet for potential drainage coming from the fistula tract. In some preferred embodiments, care is taken to not block or otherwise close a fistula opening to facilitate drainage of the tract following a grafting procedure, for example, during remodeling when a remodelable material is utilized in a grafting assembly.

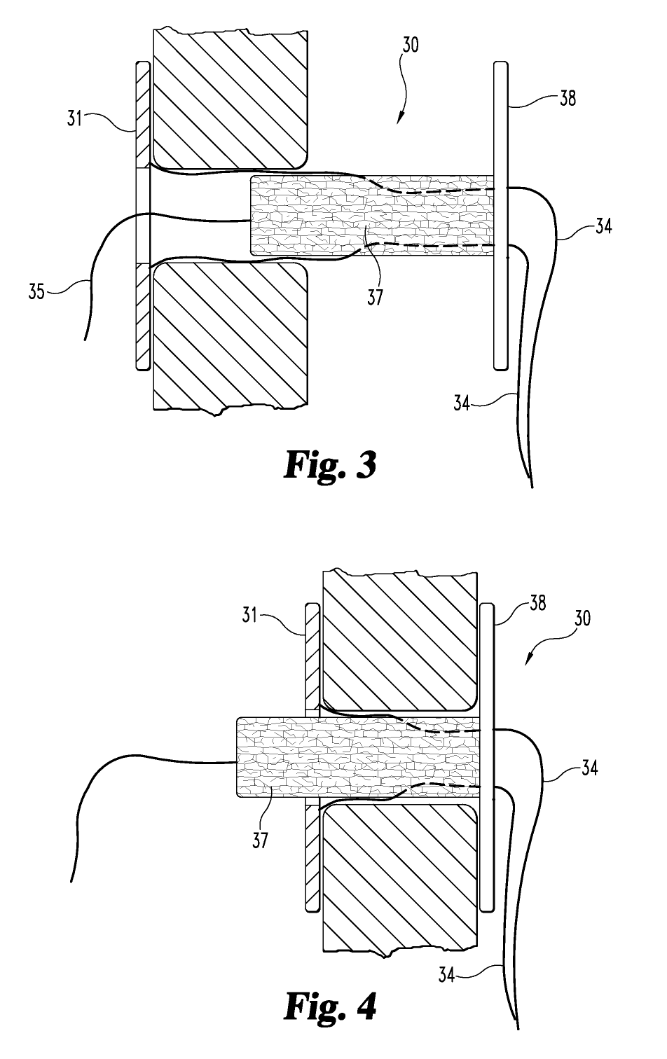

FIG. 3 shows the fistula graft assembly from FIG. 1 except additionally incorporating an elongate plug body 37, which extends from a second capping member 38. First capping member 31 is now shown in cross-section. Plug body 37 includes a generally cylindrical construct including a rolled and compressed sheet-form ECM material, although it could be otherwise shaped and configured as described elsewhere herein. Second capping member 38 and plug body 37 may be formed together as a single unit, or alternatively, the two members may be formed separately and then combined with one another. In some forms, such components are formed as separate constructs, and then coupled to one another with an absorbable coupling device or material (e.g., an adhesive).

Degradable and nondegradable coupling devices can exhibit any suitable size, shape, and configuration, and in some embodiments, take the form of one or more hooks, fasteners, barbs, straps, suture strands, or combinations thereof. Degradable coupling elements may be comprised of one or more of a variety of suitable biocompatible materials exhibiting a rate of degradation upon implantation in vivo, such as but not limited to a 2-0 vicryl suture material. Illustratively, a coupling element can be adapted to desirably hold a capping member and plug body in association with another during product handling and implantation, and then upon implantation, to degrade at a desirable rate. In some modes of operation, the capping member and elongate plug body, at least due in part to degradation of the coupling element, can uncouple or otherwise disengage from one another after a period of time following implantation, allowing the capping member to pass through and out of the body.

Continuing with FIG. 3, second pulling member 35 can be bonded or coupled to plug body 37, and thereafter, used to pull plug body 37 into fistula tract 22 through second fistula opening 22 as shown. In the tract, the sutures of first pulling member 34 can extend along plug body 37. As force is applied to second pulling member 35, a contemporaneous force may be applied to first pulling member 34, for example, to pull first capping member 31 to a position over first fistula opening 21 as shown. While not necessary to broader aspects of the invention, in some forms, second capping member will be adapted so that the sutures of first pulling member 34 can pass through the capping member, for example, through one or more openings in the capping member. Additionally, a plug body such as plug body 37 may be adapted so that a suture or other suitable pulling member can pass through a portion of the plug body along all or part of its length (e.g., through a central lumen optionally provided in the plug body, through a channel extending into the plug body from a surface of the body, etc.). In the current embodiment, each suture of first pulling member 34 passes through a volume of plug body 37 in a region of the body proximate capping member 38, for example, through a crevice, channel or other passage in the body. Illustratively, an interior channel can have one opening in an exterior side wall of the body and another opening in an exterior end wall of the plug body.

Force can be applied to first pulling member 34 to generally maintain first capping member 31 over first fistula opening 20, while a contemporaneous force is applied to second pulling member 35 to further advance pull plug body 37 through fistula tract 22. Referring now to FIG. 4, plug body 37 can be positioned in fistula tract 22 as shown, with plug body 37 extending through central opening 32 in first capping member 31, and with second capping member 38 positioned over second fistula opening 21. Once the components of assembly 30 are desirably arranged, steps can be taken to maintain the assembly in this general condition. Assembly components can be secured to each other and/or surrounding tissues. Illustratively, the sutures of first pulling member 34 can be pulled taught and tied off or otherwise affixed to one another (e.g., using a clip) on the side of second capping member 38 opposite fistula tract 22. Additionally or alternatively, these sutures can be bonded or coupled to second capping member 38. Optionally, steps may be taken to bond or otherwise attach second capping member 38 and/or first pulling member 34 to patient tissue at or near second fistula opening 21 although it is to be understood that in some forms of the invention no part of assembly 30 is attached to patient tissue during the placement procedure. In some instances, it is advantageous to not have to suture or otherwise attach the assembly components to patient tissue at the treatment site, for example, in instances where access to suitable anchoring tissue is poor or nonexistent, where the surrounding tissue is weakened or otherwise does not provide a desirable attachment point. Other potential advantages include quicker implant times, more consistent seating of the graft at the treatment site, and avoiding the possible failure of sutures, adhesives or other anchoring devices or materials that might otherwise be used to seat the graft at the treatment site.

Optionally, parts of assembly 30 in and/or around first fistula opening 20 can be manipulated in an effort to maintain the assembly in a desirable condition for providing treatment. Illustratively, plug body 37 can be directly or indirectly attached to first capping member 31, although embodiments in which a plug body is left unattached to a capping member through which it extends are contemplated as within the scope of the present invention. In this regard, an unattached plug body will be free to move back and forth through the capping member which in some forms will be effective to provide a type of strain relief to the assembly. In addition, parts of plug body 37 can be altered or removed during a placement procedure. Illustratively, any part of the plug body extending out from central opening 32 can be severed from the remainder of the plug body and discarded. Optionally, the remaining plug body portion can then be attached to first capping member 31 and/or patient tissue at or near first fistula opening 20.

In positioning the different assembly components, varying degrees of force can be applied to first pulling member 34 and second pulling member 35. Thus, it is possible to apply a considerable amount of force to the pulling members, and in some instances, the forces will be applied contemporaneously and will be sufficient to shorten the distance between first capping member 31 and second capping member 38 at the treatment site relative to the distance that would be measured under minimal tension. The assembly can then be generally maintained in this condition to provide treatment, for example, by securing the sutures to other parts of the assembly and/or patient tissue, or as otherwise described herein.

Turning now to a more detailed discussion of some of the materials that are useful in forming graft constructs of the invention, these materials should generally be biocompatible, and in advantageous embodiments of the products, are comprised of a remodelable material. Particular advantage can be provided by medical products including a remodelable collagenous material. Such remodelable collagenous materials, whether reconstituted or non-reconstituted, can be provided, for example, by collagenous materials isolated from a warm-blooded vertebrate, and especially a mammal. Such isolated collagenous material can be processed so as to have remodelable, angiogenic properties and promote cellular invasion and ingrowth. Remodelable materials may be used in this context to promote cellular growth on, around, and/or within tissue in which a medical graft product of the invention is implanted, e.g., around tissue defining a fistula tract or an opening to a fistula.

Suitable remodelable materials can be provided by collagenous extracellular matrix (ECM) materials possessing biotropic properties. For example, suitable collagenous materials include ECM materials such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa including small intestinal submucosa, stomach submucosa, urinary bladder submucosa, and uterine submucosa. Submucosa useful in the present invention can be obtained by harvesting such tissue sources and delaminating the submucosa from smooth muscle layers, mucosal layers, and/or other layers occurring in the tissue source. For additional information as to submucosa useful in the present invention, and its isolation and treatment, reference can be made, for example, to U.S. Pat. Nos. 4,902,508, 5,554,389, 5,993,844, 6,206,931, and 6,099,567.

Submucosa or other ECM tissue used in the invention is preferably highly purified, for example, as described in U.S. Pat. No. 6,206,931 to Cook et al. Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of any ECM tissue used in the present invention.

A typical layer thickness for an as-isolated submucosa or other ECM tissue layer used in the invention ranges from about 50 to about 250 microns when fully hydrated, more typically from about 50 to about 200 microns when fully hydrated, although isolated layers having other thicknesses may also be obtained and used. These layer thicknesses may vary with the type and age of the animal used as the tissue source. As well, these layer thicknesses may vary with the source of the tissue obtained from the animal source.

Suitable bioactive agents may include one or more bioactive agents native to the source of the ECM tissue material. For example, a submucosa or other remodelable ECM tissue material may retain one or more growth factors such as but not limited to basic fibroblast growth factor (FGF-2), transforming growth factor beta (TGF-beta), epidermal growth factor (EGF), cartilage derived growth factor (CDGF), and/or platelet derived growth factor (PDGF). As well, submucosa or other ECM materials when used in the invention may retain other native bioactive agents such as but not limited to proteins, glycoproteins, proteoglycans, and glycosaminoglycans. For example, ECM materials may include heparin, heparin sulfate, hyaluronic acid, fibronectin, cytokines, and the like. Thus, generally speaking, a submucosa or other ECM material may retain one or more bioactive components that induce, directly or indirectly, a cellular response such as a change in cell morphology, proliferation, growth, protein or gene expression.

Submucosa or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination with appropriate staining. Such non-collagenous solids can constitute a significant percentage of the dry weight of the ECM material in certain inventive embodiments, for example at least about 1%, at least about 3%, and at least about 5% by weight in various embodiments of the invention.

The submucosa or other ECM material used in the present invention may also exhibit an angiogenic character and thus be effective to induce angiogenesis in a host engrafted with the material. In this regard, angiogenesis is the process through which the body makes new blood vessels to generate increased blood supply to tissues. Thus, angiogenic materials, when contacted with host tissues, promote or encourage the formation of new blood vessels into the materials. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of such native bioactive components, non-native bioactive components such as those synthetically produced by recombinant technology or other methods (e.g., genetic material such as DNA), may be incorporated into an ECM material. These non-native bioactive components may be naturally-derived or recombinantly produced proteins that correspond to those natively occurring in an ECM tissue, but perhaps of a different species. These non-native bioactive components may also be drug substances. Illustrative drug substances that may be added to materials include, for example, anti-clotting agents, e.g. heparin, antibiotics, anti-inflammatory agents, thrombus-promoting substances such as blood clotting factors, e.g., thrombin, fibrinogen, and the like, and anti-proliferative agents, e.g. taxol derivatives such as paclitaxel. Such non-native bioactive components can be incorporated into and/or onto ECM material in any suitable manner, for example, by surface treatment (e.g., spraying) and/or impregnation (e.g., soaking), just to name a few. Also, these substances may be applied to the ECM material in a premanufacturing step, immediately prior to the procedure (e.g., by soaking the material in a solution containing a suitable antibiotic such as cefazolin), or during or after engraftment of the material in the patient.

Medical graft products of the invention can include xenograft material (i.e., cross-species material, such as tissue material from a non-human donor to a human recipient), allograft material (i.e., interspecies material, with tissue material from a donor of the same species as the recipient), and/or autograft material (i.e., where the donor and the recipient are the same individual). Further, any exogenous bioactive substances incorporated into an ECM material may be from the same species of animal from which the ECM material was derived (e.g. autologous or allogenic relative to the ECM material) or may be from a different species from the ECM material source (xenogenic relative to the ECM material). In certain embodiments, ECM material will be xenogenic relative to the patient receiving the graft, and any added exogenous material(s) will be from the same species (e.g. autologous or allogenic) as the patient receiving the graft. Illustratively, human patients may be treated with xenogenic ECM materials (e.g. porcine-, bovine- or ovine-derived) that have been modified with exogenous human material(s) as described herein, those exogenous materials being naturally derived and/or recombinantly produced.

ECM materials used in the invention may be free of additional, non-native crosslinking, or may contain additional crosslinking. Such additional crosslinking may be achieved by photo-crosslinking techniques, by chemical crosslinkers, or by protein crosslinking induced by dehydration or other means. However, because certain crosslinking techniques, certain crosslinking agents, and/or certain degrees of crosslinking can destroy the remodelable properties of a remodelable material, where preservation of remodelable properties is desired, any crosslinking of the remodelable ECM material can be performed to an extent or in a fashion that allows the material to retain at least a portion of its remodelable properties. Chemical crosslinkers that may be used include for example aldehydes such as glutaraldehydes, diimides such as carbodiimides, e.g., 1-ethyl-3-(3-dimethylaminopropyl)carbodiimide hydrochloride, ribose or other sugars, acyl-azide, sulfo-N-hydroxysuccinamide, or polyepoxide compounds, including for example polyglycidyl ethers such as ethyleneglycol diglycidyl ether, available under the trade name DENACOL EX810 from Nagese Chemical Co., Osaka, Japan, and glycerol polyglycerol ether available under the trade name DENACOL EX 313 also from Nagese Chemical Co. Typically, when used, polyglycerol ethers or other polyepoxide compounds will have from 2 to about 10 epoxide groups per molecule.

Turning now to a discussion of drying techniques that can be useful in certain embodiments of the invention, drying by evaporation, or air drying, generally comprises drying a partially or completely hydrated remodelable material by allowing the hydrant to evaporate from the material. Evaporative cooling can be enhanced in a number of ways, such as by placing the material in a vacuum, by blowing air over the material, by increasing the temperature of the material, by applying a blotting material during evaporation, or by any other suitable means or any suitable combination thereof. The amount of void space or open matrix structure within an ECM material that has been dried by evaporation is typically more diminished than, for example, an ECM material dried by lyophilization as described below.

A suitable lyophilization process can include providing an ECM material that contains a sufficient amount of hydrant such that the voids in the material matrix are filled with the hydrant. The hydrant can comprise any suitable hydrant known in the art, such as purified water or sterile saline, or any suitable combination thereof. Illustratively, the hydrated material can be placed in a freezer until the material and hydrant are substantially in a frozen or solid state. Thereafter, the frozen material and hydrant can be placed in a vacuum chamber and a vacuum initiated. Once at a sufficient vacuum, as is known in the art, the frozen hydrant will sublime from the material, thereby resulting in a dry remodelable material.

In alternative embodiments, a hydrated ECM material can be lyophilized without a pre-freezing step. In these embodiments, a strong vacuum can be applied to the hydrated material to result in rapid evaporative cooling which freezes the hydrant within the ECM material. Thereafter, the frozen hydrant can sublime from the material thereby drying the ECM material. Desirably, an ECM material that is dried via lyophilization maintains a substantial amount of the void space, or open matrix structure, that is characteristic of the harvested ECM material.

Drying by vacuum pressing generally comprises compressing a fully or partially hydrated remodelable material while the material is subject to a vacuum. One suitable method of vacuum pressing comprises placing a remodelable material in a vacuum chamber having collapsible walls. As the vacuum is established, the walls collapse onto and compress the material until it is dry. Similar to evaporative drying, when a remodelable material is dried in a vacuum press, more of the material's open matrix structure is diminished or reduced than if the material was dried by lyophilization.

In certain aspects, the invention provides medical products including a multilaminate material. Such multilaminate materials can include a plurality of ECM material layers bonded together, a plurality of non-ECM materials bonded together, or a combination of one or more ECM material layers and one or more non-ECM material layers bonded together. To form a multilaminate ECM material, for example, two or more ECM segments are stacked, or one ECM segment is folded over itself at least one time, and then the layers are fused or bonded together using a bonding technique, such as chemical cross-linking or vacuum pressing during dehydrating conditions. An adhesive, glue or other bonding agent may also be used in achieving a bond between material layers. Suitable bonding agents may include, for example, collagen gels or pastes, gelatin, or other agents including reactive monomers or polymers, for example cyanoacrylate adhesives. As well, bonding can be achieved or facilitated between ECM material layers using chemical cross-linking agents such as those described above. A combination of one or more of these with dehydration-induced bonding may also be used to bond ECM material layers to one another.

A variety of dehydration-induced bonding methods can be used to fuse together portions of an ECM material. In one preferred embodiment, multiple layers of ECM material are compressed under dehydrating conditions. In this context, the term “dehydrating conditions” is defined to include any mechanical or environmental condition which promotes or induces the removal of water from the ECM material. To promote dehydration of the compressed ECM material, at least one of the two surfaces compressing the matrix structure can be water permeable. Dehydration of the ECM material can optionally be further enhanced by applying blotting material, heating the matrix structure or blowing air, or other inert gas, across the exterior of the compressed surfaces. One particularly useful method of dehydration bonding ECM materials is lyophilization.

Another method of dehydration bonding comprises pulling a vacuum on the assembly while simultaneously pressing the assembly together. Again, this method is known as vacuum pressing. During vacuum pressing, dehydration of the ECM materials in forced contact with one another effectively bonds the materials to one another, even in the absence of other agents for achieving a bond, although such agents can be used while also taking advantage at least in part of the dehydration-induced bonding. With sufficient compression and dehydration, the ECM materials can be caused to form a generally unitary ECM structure.

It is advantageous in some aspects of the invention to perform drying and other operations under relatively mild temperature exposure conditions that minimize deleterious effects upon any ECM materials being used, for example native collagen structures and potentially bioactive substances present. Thus, drying operations conducted with no or substantially no duration of exposure to temperatures above human body temperature or slightly higher, say, no higher than about 38° C., will preferably be used in some forms of the present invention. These include, for example, vacuum pressing operations at less than about 38° C., forced air drying at less than about 38° C., or either of these processes with no active heating—at about room temperature (about 25° C.) or with cooling. Relatively low temperature conditions also, of course, include lyophilization conditions.

Additionally, medical products of the invention may include biocompatible materials derived from a number of biological polymers, which can be naturally occurring or the product of in vitro fermentation, recombinant genetic engineering, and the like. Purified biological polymers can be appropriately formed into a substrate by techniques such as weaving, knitting, casting, molding, and extrusion. Suitable biological polymers include, without limitation, collagen, elastin, keratin, gelatin, polyamino acids, polysaccharides (e.g., cellulose and starch) and copolymers thereof. As well, any portion of an inventive product can also be formed with a suitable synthetic polymeric material including but not limited to the bioresorbable and/or non-bioresorbable plastics described elsewhere herein.

The present invention also provides, in certain aspects, medical products that include a radiopaque element such as but not limited to a radiopaque coating, attached radiopaque object, or integrated radiopaque substance. In this regard, a capping member and/or a elongate plug body of some inventive assemblies may be comprised of a radiopaque element. Any suitable radiopaque substance, including but not limited to, tantalum such as tantalum powder, can be incorporated into a medical product of the invention. Other radiopaque materials comprise bismuth, iodine, and barium, as well as other suitable markers.

In certain aspects, the invention provides graft assemblies incorporating an expandable element (e.g., an expandable material and/or device). In this regard, an inventive assembly may be provided, wherein all or part of a capping member, and if present, an elongate plug body, have the capacity to expand. For example, a graft product can include, for example, a suitable ECM foam or sponge form material. Illustratively, a graft assembly, or any portion thereof, may comprise a porous, three-dimensionally stable body formed with one or more suitable biocompatible matrix materials. Such biocompatible matrix materials can include naturally-occurring polymers and/or synthetic polymers. More preferred sponge compositions will comprise collagen as a matrix-forming material, either alone or in combination with one or more other matrix forming materials, and particularly preferred sponge compositions will comprise an ECM material such as those discussed elsewhere herein. In general, sponge matrices useful in certain embodiments of the present invention can be formed by providing a liquid solution or suspension of a matrix-forming material, and causing the material to form a porous three-dimensionally stable structure; however, a sponge or foam material can be formed using any suitable formation method, as is known in the art. For additional information concerning foam or sponge form materials that can be useful in certain embodiments of the present invention, reference can be made, for example, to U.S. Pat. App. Pub. No. 2003/0013989.

In some forms, a compact, stabilized sponge construct is highly expansive when wetted, which can desirably enhance the ability of the construct to fill at least part of a fistula. In illustrative procedures, a suitable hydrant, such as saline, may be applied or delivered to a sponge construct after it is suitably located within a patient to enhance the expansion of the construct within the fistula tract and/or a fistula opening. Alternatively or additionally, a bodily fluid of the patient can sufficiently wet the implanted graft construct so as to promote the expansion of the construct within the fistula.

In certain forms of the invention, a graft product incorporates an anchoring adaptation to maintain the product in a desirable position at the treatment site following product implantation. For example, a medical product can include an adhesive for maintaining contact in and/or around the fistula. An adhesive can be applied to the graft product before an implantation procedure, e.g., during manufacture of the product, or alternatively, can be applied to the graft product and/or to tissue at or near the fistula during such an implantation procedure. Other suitable anchoring adaptations include but are not limited to barbs, hooks, sutures, protuberances, ribs, and the like. Again, such anchoring adaptations, while advantageous in certain inventive embodiments are not necessary to broader aspects of the invention. Illustratively, certain medical graft products are configured so that a capping member is able to maintain contact with portions of a bodily tissue wall adjacent to a fistula opening following implantation without the need for such anchoring adaptations. In other aspects, suitable anchoring adaptations aid or facilitate the maintenance of such contact.

Additionally, in some illustrative embodiments, one or more anchors, barbs, ribs, protuberances, and/or other suitable surface modifications can be incorporated on and/or within an illustrative plug body to roughen, condition, or otherwise de-epithelialize at least a portion of the fistula, such as the fistula tract and/or the primary opening, during and/or after emplacement of the plug within the tract. The conditioning of the tract tissue can serve to initiate a localized healing response in patient tissue that can be advantageous in enhancing the ingrowth of patient tissue into an illustrative plug construct, such as a plug comprising an ECM material. Further, in illustrative embodiments, where a suture, leader, or string is used to assist with the emplacement of an illustrative graft construct within a tract, as is discussed elsewhere herein, the leader can comprise an abrasive material, or comprise one or more sections and/or surface features and/or adaptations, e.g. one or more bristles that can directionally emanate from the leader material and that can serve to roughen or otherwise condition or de-epithelialize patient tissue upon travel through and/or location within a fistula tract.

In certain aspects, medical graft products of the invention incorporate an adhesive or, where appropriate, a sclerosing agent to facilitate and/or promote blocking of at least the primary opening of the fistula. As well, fistula treatment methods of the invention can include steps where such substances or materials are applied to a medical graft product being deployed and/or to the soft tissues surrounding the fistula. For example, an adhesive, glue or other bonding agent may also be used in achieving a bond between a medical graft product of the invention and the soft tissues defining a fistula opening or tract and/or adjacent tissues. Suitable bonding agents may include, for example, fibrin or collagen gels or pastes, gelatin, or other agents including reactive monomers or polymers, e.g., cyanoacrylate adhesives. In some forms of the invention, a fistula treatment method includes contacting soft tissue surfaces surrounding the fistula, e.g., soft tissue surfaces at or near the primary opening and/or soft tissues lining the fistula tract, with a sclerosing agent prior to forcing the sheet from material into the fistula. Such use of a sclerosing agent can de-epithelialize or otherwise damage or disrupt these soft tissue surfaces, leading to the initiation of a healing response.

In some forms, a fistula is drained prior to receiving a medical graft product of the invention therein. Such draining can be accomplished by inserting a narrow diameter rubber drain known as a seton (Greek, “thread”) through the fistula. The seton is passed through the fistula tract and tied as a loop around the contained tissue and left for several weeks or months, prior to definitive closure or sealing of the fistula. This procedure is usually performed to drain infection from the area, and to mature the fistula tract prior to a definitive closure procedure.

Additionally, the present invention provides kits that include products as described herein for treating fistulae, e.g., in sterile medical packaging. The kits can include written materials including instructions for delivering and/or otherwise using the products to treat fistulae, e.g., to treat rectovaginal fistulae as described herein. Related embodiments of the invention include methods for distributing such products for treating fistulae, or otherwise conducting business, which include distributing such products for treating fistulae, and also distributing information relating the use of such products for treating fistulae. Such information can be distributed packaged with the products for treating fistula, or separately, e.g., including information or instructions available on a communication network, including a global computer communication network such as the internet.

Some embodiments of the invention provide a line of medical kits, wherein a medical kit of the invention includes one or more products of the invention in a sealed package. In some forms of the invention, medical kits are provided that include one or more fistula treatment products such as any of those described herein, and potentially also suitable instrumentation to be used in the delivery of the product to the treatment site, enclosed within sterile medical packaging. Illustratively, such a medical product can have packaging including a backing layer and a front film layer that are joined by a boundary of pressure-adhesive as is conventional in medical packaging, wherein the contents of the packaging are sealed between the backing layer and front film layer. Sterilization of such a medical product may be achieved, for example, by irradiation, ethylene oxide gas, or any other suitable sterilization technique, and the materials and other properties of the medical packaging will be selected accordingly. Additionally, the packaging can include indicia to communicate the contents of the package to a person, machine, computer, and/or electronic device. Such indicia may include the dimensions of, the type of materials used to form, and/or other useful information relating to the contents of the package.

Fistula treatment methods of the invention may include an endoscopic visualization (fistuloscopy) step. Such endoscopic visualization can be used, for example, to determine the shape and size of the fistula, which in turn can be used to select an appropriately sized and shaped medical graft product for treating the fistula. Illustratively, a very thin flexible endoscope can be inserted into a secondary opening of the fistula and advanced under direct vision through the fistula tract and out through the primary opening. By performing fistuloscopy of the fistula, the primary opening can be accurately identified. Also, cleaning of the fistula can be performed prior to and/or during deployment of a medical graft product of the invention. For example, an irrigating fluid can be used to remove any inflammatory or necrotic tissue located within the fistula prior to engrafting the product. In certain embodiments, one or more antibiotics are applied to the medical graft product and/or the soft tissues surrounding the fistula as an extra precaution or means of treating any residual infection within the fistula.

Additionally, the medical graft products of the invention can be modified before, during, and/or after deployment. Illustratively, a product may be cut, trimmed, sterilized, and/or treated (e.g., brought into contact, impregnated, coated, etc.) with one or more desirable compositions, such as any of those previously disclosed herein, e.g., anticoagulants (e.g., heparin), growth factors or other desirable property modifiers. In certain aspects, following deployment of a graft assembly in accordance with the present invention, one or more portions of the assembly are modified, for example, trimmed off or otherwise removed, for example, material protruding from the primary opening and/or any secondary opening.

In certain aspects, a plug body is utilized and comprises a material receptive to tissue ingrowth. In such aspects, upon deployment of the body in accordance with the present invention, cells from the patient can infiltrate the body material, leading to, for example, new tissue growth on, around, and/or within the material. In some embodiments, the medical graft product comprises a remodelable material. In these embodiments, the remodelable material promotes and/or facilitates the formation of new tissue, and is capable of being broken down and replaced by new tissue in such a way that the original fistula closure achieved by the implanted plug body is maintained throughout the remodeling process so as to eventually form a closure or substantial closure with the new tissue.

Further, the fistula treatment methods described herein can be used to close one or more fistula during a given medical procedure. Also, methods of the invention can be used to treat complex fistula. For multiple fistula, multiple medical graft products can be engrafted until all the fistula have been addressed. In cases of complex fistula, for example a horse-shoe fistula, there may be one primary opening and two or more fistula tracts extending from that opening. In such instances, a medical graft product may be configured with multiple capping members and multiple plug bodies.

Additional embodiments of the invention provide methods for treating fistulae that involve the use of flowable remodelable extracellular matrix material. In such embodiments, the flowable material can be used to fill openings and/or tracts of fistulae, including anorectal or other alimentary fistulae, and promote tissue ingrowth to close the fistulae. In this regard, the flowable material can be delivered in any suitable fashion, including for example forcible ejection from cannulated members such as catheters, sheaths, or needles. Suitable flowable, remodelable ECM materials for use in this aspect of the invention can be prepared, for example, as described in U.S. Pat. Nos. 5,275,826 and 5,516,533 or in International Publication No. WO2005020847 (Cook Biotech Incorporated) published Mar. 10, 2005, which are each hereby incorporated by reference in their entirety. Such flowable materials can include solubilized and/or particulate ECM components, and in preferred forms include ECM gels having suspended therein ECM particles, for example having an average particle size of about 50 microns to about 500 microns, more preferably about 100 microns to about 400 microns. The ECM particulate can be added in any suitable amount relative to the solubilized ECM components, with preferred ECM particulate to ECM solubilized component weight ratios (based on dry solids) being about 0.1:1 to about 200:1, more preferably in the range of 1:1 to about 100:1. The inclusion of such ECM particulates in the ultimate gel can serve to provide additional material that can function to provide bioactivity to the gel (e.g. itself including FGF-2 and/or other growth factors or bioactive substances as discussed herein) and/or serve as scaffolding material for tissue ingrowth. Flowable ECM materials can also be used in conjunction with graft assemblies as described herein. Implanted assemblies can, for example, be provided at a fistula treatment location, and can act as a confining barrier to an amount of flowable ECM material introduced against the barrier and filling the tract of the fistula to promote healing.

All publications and patent applications cited in this specification are herein incorporated by reference as if each individual publication or patent application were specifically and individually indicated to be incorporated by reference. Further, any theory, mechanism of operation, proof, or finding stated herein is meant to further enhance understanding of the present invention, and is not intended to limit the present invention in any way to such theory, mechanism of operation, proof, or finding. While the invention has been illustrated and described in detail in the drawings and foregoing description, the same is to be considered as illustrative and not restrictive in character, it being understood that only selected embodiments have been shown and described and that all equivalents, changes, and modifications that come within the spirit of the inventions as defined herein or by the following claims are desired to be protected.

What is claimed is:

1. A fistula graft assembly for treating a fistula having at least a first fistula opening, a second fistula opening and a fistula tract extending therebetween, the assembly comprising:
a first capping member positionable over the first fistula opening and having a first pulling member extending therefrom;
a second capping member positionable over the second fistula opening and having a second pulling member extending therefrom;
an elongate plug body configured to reside in the fistula tract,
wherein the first pulling member and the second pulling member are configured to extend along the plug body in the fistula tract, and can be contemporaneously pulled and in generally opposite directions for maintaining the first capping member over the first fistula opening and the second capping member over the second fistula opening, respectively.

2. The fistula graft assembly of claim 1, wherein the elongate plug body includes a portion configured to extend the entire length of the fistula tract.

3. The fistula graft assembly of claim 1, wherein at least one of said first capping member and said second capping member comprises a naturally derived material.

4. The fistula graft assembly of claim 1, wherein at least one of said first capping member and said second capping member comprises a synthetic polymeric material.

5. The fistula graft assembly of claim 1, wherein at least one of said first capping member and said second capping member includes a support frame.

6. The fistula graft assembly of claim 1, wherein at least one of said first capping member and said second capping member comprises an expandable element.

7. The fistula graft assembly of claim 1, wherein the elongate plug body comprises a collagenous material.

8. The fistula graft assembly of claim 1, wherein the elongate plug body comprises a remodelable material.

9. The fistula graft assembly of claim 1, wherein the elongate plug body comprises an extracellular matrix material.

10. The fistula graft assembly of claim 9, wherein said extracellular matrix material comprises submucosa, serosa, pericardium, dura mater, peritoneum, or dermal collagen.

11. The fistula graft assembly of claim 1, wherein at least one of said first capping member and said second capping member provides an opening through which said elongate plug body can extend.

12. The fistula graft assembly of claim 1, wherein the elongate plug body includes a tapered portion.

13. The fistula graft assembly of claim 1, wherein said elongate plug body provides a passage through which at least one of said first pulling member and said second pulling member can be passed.

14. The fistula graft assembly of claim 1, wherein at least one of said first pulling member and said second pulling member comprises a suture.

15. A fistula graft assembly for treating a fistula having at least a first fistula opening, a second fistula opening and a fistula tract extending therebetween, comprising:
a first capping member positionable over the first fistula opening and having a first pulling member extending therefrom; and
an elongate plug body configured to reside in the fistula tract and including a portion configured to extend through an opening in the first capping member,
wherein the first pulling member is configured to extend through the fistula tract, and is pullable in a direction generally away from the first fistula opening for maintaining the first capping member over the first fistula opening.

16. The fistula graft assembly of claim 15, wherein one or more slits in the first capping member provides said opening.

17. The fistula graft assembly of claim 15, further comprising a second capping member to be positioned over the second fistula opening.

18. The fistula graft assembly of claim 17, wherein the second capping member is configured for translation along said elongate plug body.

19. The fistula graft assembly of claim 18, wherein the second capping member is configured for receipt over said elongate plug body.

20. A method of treating a fistula having at least a first fistula opening, a second fistula opening and a fistula tract extending therebetween, comprising
providing a first capping member having a first pulling member extending therefrom;
providing a second capping member having a second pulling member extending therefrom;
positioning the first capping member over the first fistula opening, wherein the first pulling member extends through the fistula tract;
positioning the second capping member over the second fistula opening, wherein the second pulling member extends through the fistula tract; and
applying a first pulling force to the first pulling member for maintaining the first capping member over the first fistula opening; and
applying a second pulling force to the second pulling member and contemporaneous with the first pulling force for maintaining the second capping member over the second fistula opening.

21. The method of claim 20, wherein at least one of said first pulling member and said second pulling member comprises an elongate plug body.

22. A method of treating a fistula having at least a first fistula opening, a second fistula opening and a fistula tract extending therebetween, the method comprising
providing a first capping member having a first pulling member extending therefrom;
positioning the first capping member over the first fistula opening such that the first pulling member extends through the fistula tract;
providing an elongate plug body; and
positioning the plug body in the fistula tract, wherein the plug body is advanced through the fistula tract from the second fistula opening and toward the first fistula opening.
